(19) United States
(12) Patent Application Publication
(10) Pub. No.: US 2010/0252779 A1
(43) Pub. Date: Oct. 7, 2010

(54) COPPER BROMIDE HUMIDITY INDICATING CARD

(75) Inventor: George E. McKedy, Williamsville, NY (US)

Correspondence Address: Stephen B. Salai, Esq.;Harter Secrest & Emery LLP, 1600 Bausch & Lomb Place, Rochester, NY, 14604-2711, US

(73) Assignee: MULTISORB TECHNOLOGIES, INC., Buffalo, NY (US)

(21) Appl. No.: 12/419,116

(22) Filed: Apr. 6, 2009

(51) Int. Cl. G01N 33/00

(52) U.S. Cl. 252/408.1

(57) ABSTRACT

The present invention comprises a method of humidity indication comprising providing a substrate and an area of humidity indication comprising copper bromide, bromide salt, a dye, and cellulose fiber. In another embodiment of the invention, the invention comprises a humidity indicator array comprising a substrate having a plurality of areas of humidity indication on the substrate, wherein each area comprises copper bromide, dye, and bromide salt, wherein the mole ratio of bromide ions to copper ions in the area is about 7.5:1.

### BACKGROUND OF THE INVENTION

1. Field of the Invention

The present invention relates to humidity indicators used for indicating the relative humidity in an environment, and more particularly, to copper bromide humidity indicators which change color at different relative humidities in an environment.

2. Description of Related Art

A variety of humidity indicators exist today. One type of humidity indicator is a humidity indicating card, which is used for many purposes including, determining the relative humidity in shipping containers and packages, such as packaged electronics and telecommunication products. The humidity indicator cards are typically impregnated with a humidity sensitive composition and dried. Most humidity indicating cards made today use cobalt chloride as the indicator, which changes color based on the level of hydration of the cobalt chloride. Cobalt chloride can change from a blue color, when it has one water of hydration, to a pink color when it has six waters of hydration. That is, the blue color becomes increasingly red with each additional water of hydration. Therefore, one can determine the approximate humidity based on the color present on the card. An array of humidity indication areas can be included on the card, wherein increasing humidity levels are required to change different areas of indication on the card from blue to pink.

A problem with humidity indicators using cobalt chloride is that the cobalt chloride is a known irritant and a toxin. Further, the use of cobalt chloride is subject to regulation when used with food and pharmaceutical products. Thus, cobalt chloride humidity indicators have limited applications and are not useful in the food and pharmaceutical industries.

Some types of humidity indicator cards have impregnated halides or other salts capable of releasing Cu²⁺ ions. International Publication No. WO 02/44712 discloses a humidity indicator paper impregnated with copper salts, a synergistic salt, and a dye.

Other types of humidity indicators include the use of humidity indicating gels. In US Patent Application Publication No. 2004/0051081, Moreton discloses a silica gel material impregnated with copper and bromide to provide a relative humidity indicator. In U.S. Pat. No. 6,655,315 Gattiglia discloses a silica gel impregnated with copper chloride and hygroscopic salts, wherein the silica gel changes color to indicate the presence of moisture.

There remains a need for an improved humidity indicator that provides easily detectible color changes and that is non-toxic.

### BRIEF SUMMARY OF THE INVENTION

The present invention comprises a method of humidity indication comprising providing a substrate and an area of humidity indication comprising copper bromide, bromide salt, a dye, and cellulose fiber.

In another embodiment of the invention, the invention comprises a humidity indicator array comprising a substrate having a plurality of areas of humidity indication on the substrate, wherein each area comprises copper bromide, dye, and bromide salt, wherein the mole ratio of bromide ions to copper ions in the area is about 7.5:1.

### BRIEF DESCRIPTION OF THE SEVERAL VIEWS OF THE DRAWING(S)

FIG. 1 is a top view of a humidity indicator card of the present invention.

### DETAILED DESCRIPTION OF THE INVENTION

This invention overcomes disadvantages of prior practices. The present invention provides a non-toxic humidity indicator. The invention also provides a device that can be used in food and pharmaceutical product packages. Further, the invention provides a device that indicates when a desiccant needs to be changed. The invention also provides a color change in areas of a card or other substrate that is easily detectable by a user. Further, the invention allows one to vary the color of the indicator areas. These and other advantages will be apparent from the detailed description below.

The present invention includes a humidity indicating device having at least one humidity indicating area. In a preferred form, the present invention includes a humidity indicator array comprising a plurality of areas of humidity indication on a substrate. The humidity indication areas each have a composition capable of changing color at a certain relative humidity. The humidity indicating composition includes copper bromide, a bromide salt and a dye in effective combinations that will react with water vapor to provide [Cu(H₂0)₆]²⁺ ions.

In a preferred form, the composition can be made by mixing together copper (II) sulfate pentahydrate, sodium bromide, cellulose fiber, a dye and water. An array of solutions having increasing amounts of copper (II) pentahydrate and sodium bromide and the same amount of dye and cellulose fiber are cast onto separate areas of the substrate. The dye is generally absorbed into the paper and is only visible when the pale tan color of the [Cu(H₂0)₆]²⁺ ions is present as described below.

The copper II sulfate pentahydrate and sodium bromide react to form copper bromide complexes, which in the anhydrous form provide a dark brown appearance and mask the dye. When the copper bromide is exposed to a certain relative humidity, the water vapor molecules in the environment bind with the copper from the copper bromide complexes forming [Cu(H₂0)₆]²⁺ ions, which are pale tan in color and substantially colorless. These [Cu(H₂0)₆]²⁺ ions are not capable of masking the dye. Thus, when a certain relative humidity is reached, the presence of the [Cu(H₂0)₆]²⁺ ions imparts a color change in the humidity indicating area from the dark brown color of the copper bromide complexes to the generally invisible light tan color. The dye color is then visible. The amount of copper II sulfate and sodium bromide in the composition can be increased or decreased in each humidity indication area to control the relative humidity level at which the color change occurs. That is, increasing the amount of the copper bromide and sodium bromide present in the composition increases the relative humidity level at which the color change is observed, in part because the excess bromide in the composition competes with the water and delays the formation of the [Cu(H₂0)₆]²⁺ ions.

In some instances, it may be desirable to cast a single solution on a substrate, rather than an array of solutions, to form a single relative humidity indicating area. With a single indicating area, one can determine that the relative humidity is either above or below a particular relative humidity based on whether or not the color has changed. Alternatively, the humidity level can be determined by comparing the color of the humidity indicating area to an area on the card having a set of dyes. This set of dyes shows what color should appear at particular relative humidities. For instance, an indicating area may change color from brown to yellow when a 30% relative humidity is reached in the environment. Thus, an area on the substrate would include a yellow dye spot and a brown dye spot that can be compared to the humidity indicating area. Instead of a set of dyes, the substrate can include only one dye color, which shows the color the indicating area should be when a particular relative humidity is reached.

The preferred copper sulfate for mixing with the bromide is copper II sulfate pentahydrate because this material, when mixed with a bromide salt, forms a dark color when dry and a light tan color when a predetermined humidity level is reached, and does not react with food dyes. Other copper II sulfate materials suitable for the invention are anhydrous copper II sulfate or copper II sulfate hydrate. Typical of other copper sulfates are copper amino sulfate (copper sulfate, ammoniated) and copper sulfate tribasic.

The preferred bromide salts for mixing with copper II sulfate are sodium bromide or potassium bromide because these materials, when mixed with copper II sulfate, provide a dark color when dry and a light tan color when a predetermined humidity level is reached, and do not react with food dyes. Other bromide salts suitable for the invention are calcium bromide, magnesium bromide, zinc bromide or ammonium bromide. Surprisingly, using copper bromide as the bromide salt did not produce a color change that was as easy to detect. Other typical salts that can be used include copper chloride, copper iodine, copper fluoride, copper nitrates, chromates and permanganates.

While any type of food coloring can be used as the dye, it should be appreciated that FD&C dyes are preferred, because they are readily available and safe for use with foods, cosmetics and pharmaceuticals. The preferred dye is FD&C Yellow #5 in combination with FD&C Red #40 because it provides an easily detectible color change when a certain relative humidity is reached. Another preferred dye is FD&C Green #3 in combination with FD&C Yellow #5. In this instance, the substrate would change from brown to green when a certain relative humidity is reached. However, using only FD&C Yellow #5, FD&C Green #3, or FD&C Red #40 is suitable. It should be appreciated that other dyes, except for brown dyes, could be utilized in the invention as well.

Preferably, a cellulose solution is included in the humidity indicating solutions. For example, Klucel® hydroxypropylcellulose jf, manufactured by Hercules, Inc., can be used. Klucel® hydroxypropylcellulose jf is a powder having a particle size of 99% through a 20% mesh screen (851 microns) or 85% through a 30% mesh screen (600 microns). The cellulose solution increases the viscosity of the humidity indicating solutions, preventing the solution from migrating too far when the solution is cast onto the humidity indicating cards. It is believed that the cellulose also minimize aggregation of the copper bromide complex to provide a uniform color distribution and to prevent the formation of dark spots from the copper bromide complex. The cellulose fiber solution contains the humidity indicating solutions, such that each humidity indicating area is smaller, allowing a greater number of relative humidity detection areas to be included on the card. Further, the color changes are easier to detect, since the copper bromide complex and dye are in a more concentrated and uniform area. A preferred concentration range of cellulose fiber in the humidity indicating solutions is about 0.8%-1.2% because it provides a viscosity suitable for maintaining the copper II sulfate and bromide salt solution in a desired area on the humidity indicator card. However, a suitable range of the cellulose fiber in the humidity indicating solutions is about 0.5%-1.5%. Other thickeners are suitable to increase the viscosity of the humidity indicating solution, including carboxymethyl cellulose, carboxypropyl cellulose carrageenan, guar gum, or xanthan gum.

In a preferred embodiment of the invention, the substrate used is blotting paper because it easily absorbs the dye and dries rapidly. Although, it should be appreciated that other types of substrates may be used. For example, this humidity indicating chemistry can be used on other types of papers, adhesive stamps, sheets of plastic, films, or coating layers on a backing material, such as a polymer or fibrous coating on a substrate such as metal. In addition, the humidity indicating chemistry can be used in the solid form, or in materials, such as in resin polymers and hot melt adhesives. More specifically, polyvinyl acetate ethylene vinyl acetate, nylon, polypropylene, polyethylene, polyester, or polyacrylates can include the humidity indicating chemistry. The humidity indicating chemistry can be within the entire material or within a portion of the material. In a preferred embodiment of the invention, the material is generally transparent or colorless so the color change is more visible. This humidity indicating chemistry can also be included in a pressed part or a tablet that is made by pressing various powders or aggregates together with or without a binder.

It should be appreciated that other additives can be included in the humidity indicating solution, such as surfactants, bacteriostats and fungicides.

The color of the humidity indicating area is affected by the amount of the copper bromide and sodium bromide present in the composition. As the amount of copper bromide and sodium bromide present in the composition increases, the relative humidity level at which the color change is observed increases. Preferably, the sodium bromide to copper (II) sulfate pentahydrate in solutions for detecting a relative humidity in the range of 10% to 60% is present in an amount such that the weigh ratio is in a range from about 3:1 to 5:1. More specifically, the preferred sodium bromide to copper (II) sulfate pentahydrate weight ratio for solutions that detect a relative humidity in the range of 10% to 50% is about 3:1. The preferred sodium bromide to copper (II) sulfate pentahydrate weight ratio for solutions that detect a relative humidity above 60% is at least 5:1 because a greater excess of bromide is required to prevent the copper bromide from changing color before the 60% relative humidity level is reached.

Similarly, as the amount of copper ions and bromide ions present in the composition increases, the relative humidity level at which the color change is observed increases. It has been found that, in solutions for detecting a relative humidity in the range of 10% to 60%, the mole ratio of bromide ions to copper ions ranges from about 7.5:1 to 12.2:1. The preferred bromide ion to copper ion mole ratio for humidity indicating solutions that detect humidities ranging from 10% to 50% is about 7.5:1 and the bromide ion to copper ion mole ratio of humidity indicating solutions that detect humidities above 60% is at least 12.2:1.

It should be appreciated that this invention is well described for use in many applications, such as for determining whether a desiccant material is still active. The invention can also determine a relative humidity in product packages and containers, including food, cosmetic and pharmaceutical packages and containers, and in environments requiring a preferred relative humidity, such as laboratories, manufacturing facilities, and animal enclosures and facilities.

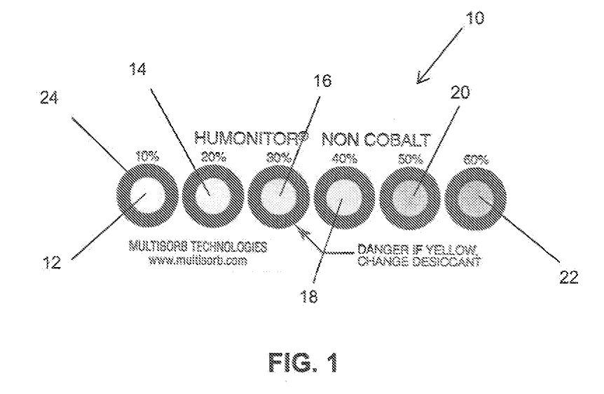

Referring now to the Figures, FIG. 1 shows a humidity indicating card 10 having six areas of relative humidity indication 12, 14, 16, 18, 20, 22. In this configuration, area 12 includes a solution having the least amount of copper II sulfate pentahydrate and sodium bromide and area 22 includes a solution having the greatest amount of copper II sulfate pentahydrate and sodium bromide. The relative humidity detection areas 12, 14, 16, 18, 20, 22 change colors from brown to yellow at increasing 10% intervals at 10%, 20%, 30%, 40%, 50% and 60%, respectively. Thus, one can bring the card 10 into an environment having humidity and determine the relative humidity in an environment by reading the highest percentage relative humidity indicator area showing the yellow dye.

Each area 12, 14, 16, 18, 20 and 22 is defined by a circle 24, which hides any solution that migrates beyond the inner area defined by the circle 24. Preferably, the circle 24 is opaque, for example, black.

For each humidity indicator area on the indicator card 10, different solutions having different concentrations of sodium bromide are made.

For, example, to create the indicator card 10 having relative humidity detection areas that detect relative humidity levels at 10%, 20%, 30%, 40%, 50% and 60%, the following solutions shown in Table 1 having copper(II) sulfate pentahydrate, sodium bromide, water and a dye solution are added in the following concentrations for the various relative humidity levels:

TABLE 1

| | Relative Humidity | | | | | |
|---|---|---|---|---|---|---|
| Ingredients (in grams) | 10% | 20% | 30% | 40% | 50% | 60% |
| Copper(II) Sulfate | 4.1 | 5.5 | 6.5 | 8.1 | 9.1 | 9.1 |
| Pentahydrate | | | | | | |
| Sodium Bromide | 12.3 | 16.5 | 19.5 | 24.3 | 27.3 | 45.5 |
| Water | 900.0 | 900.0 | 900.0 | 900.0 | 900.0 | 900.0 |
| 10% Cellulose Solution | 100.0 | 100.0 | 100.0 | 100.0 | 100.0 | 100.0 |
| Dye Solution | 9.4 | 9.4 | 9.4 | 9.4 | 9.4 | 9.4 |

The dye solution includes water, propylene glycol, FD & C Red No. 40, and FD & C Yellow #5 in the concentrations shown in Table 2 herebelow:

TABLE 2
Dye Solution

| Ingredients | Weight (in grams) |
|---|---|
| Water | 72.0 |
| Propylene glycol | 8.0 |
| FD&C Red #40 | 0.048 |
| FD&C Yellow #5 | 2.24 |

A method of making a solution for a humidity indication area for detecting a 10% relative humidity is set forth above is made as follows:
1. Add 4.1 grams of copper(II) sulfate pentahydrate, 12.3 grams of sodium bromide and 9.4 grams of the yellow dye solution to 900.0 grams of water and 100.0 grams of the 10% cellulose solution in the amount specified above for the 10% indication area; and,
2. Mix the ingredients with strong agitation for ten minutes to ensure that all of the ingredients are in solution.

The 20%-60% solutions are made in the same manner and in accordance with Table 1. Then, each solution is sprayed, blotted or pricked onto the card in the desire area. The card is then dried in a conventional oven at 300° F. for about 2-5 minutes to evaporate the water.

The copper(II) sulfate pentahydrate and the sodium bromide added to the solution form copper(II) bromide complexes when the humidity indicator card 10 is dried in the areas 12, 14, 16, 18, 20 or 22.

The 10%, 20%, 30%, 40%, 50%, and 60% solutions have increasing amounts of copper(II) sulfate pentahydrate and sodium bromide, respectively, which causes the different areas on the card to change color at different humidity levels. It should be appreciated that a 10% humidity indicating area completely changes color, from brown to yellow, at 10% humidity and will remain yellow at any humidity level above 10%. Similarly, a 50% humidity indicating area completely changes color at a 50% humidity and will remain yellow at any humidity level above 50%. The 50% humidity indicating area may begin to change color and exhibit a brown/yellow color before 50% relative humidity is reached, for example at a relative humidity of 45%, but this color change will be slight when compared to the lower humidity indicating areas.

For example, the following color changes are observed when the humidity indicating solutions set forth in Table 1 including the dye solution set forth in Table 2 are used:

TABLE 3

| Relative | | | | | | |
|---|---|---|---|---|---|---|
| Humidity | | | | | | |
| Indication | Relative Humidity | | | | | |
| Area | 15% | 25% | 35% | 45% | 55% | 65% |
| 10% | Yellow | Yellow | Yellow | Yellow | Yellow | Yellow |
| 20% | Brown | Yellow | Yellow | Yellow | Yellow | Yellow |
| 30% | Brown | Brown | Yellow | Yellow | Yellow | Yellow |
| 40% | Brown | Brown | Brown | Yellow | Yellow | Yellow |
| 50% | Brown | Brown | Brown | Brown/ | Yellow | Yellow |
| | | | | Yellow | | |
| 60% | Brown | Brown | Brown | Brown | Brown/ | Yellow |
| | | | | | Yellow | |

As indicated in the above table, the areas change from brown to yellow when their planned relative humidity level is reached. For instance, at a 15% relative humidity level, the 10% relative humidity indication area is yellow and the 20%-60% indication areas are brown. At a 35% relative humidity level, the 10%-30% relative humidity indication areas are yellow and the 40%-60% humidity indication areas are brown.

This invention being thus described, it will be obvious that the same may be varied in many ways. Such variations are not to be regarded as a departure from the spirit and scope of the invention and all such modifications are intended to be included within the scope of the following claims.

1. A humidity indicator comprising a substrate having an area of humidity indication comprising copper bromide, bromide salt, dye, and cellulous fiber.

2. The humidity indicator of claim 1, wherein the substrate is blotting paper.

3. The humidity indicator of claim 1, wherein the bromide salt is sodium bromide.

4. The humidity indicator of claim 1, wherein the cellulose fiber is at a concentration of about 1%.

5. The humidity indicator of claim 1, wherein the dye is a FD&C yellow dye.

6. The humidity indicator of claim 1, wherein the dye is a FD&C yellow dye and an FD&C red dye.

7. The humidity indicator of claim 1, wherein the copper bromide is formed from a reaction of copper(II) sulfate pentahydrate and the bromide salt.

8. The humidity indicator of claim 1, wherein the mole ratio of bromide to copper ranges from about 7.5:1 to about 12.2:1.

9. The humidity indicator of claim 7, wherein the weight ratio of sodium bromide to copper sulfate pentahydrate ranges from about 3:1 to about 5:1.

10. The humidity indicator of claim 1, wherein the copper bromide comprises a copper bromide complex.

11. The humidity indicator of claim 1, wherein the substrate is selected from a group consisting of a stamp, a sheet of plastic, a polymer film, and an absorbent coating layer on a backing material.

12. A humidity indicator array comprising a substrate having a plurality of areas of humidity indication on the substrate, wherein each area comprises copper bromide, dye, and bromide salt, wherein the mole ratio of bromide ions to copper ions in the area is about 7.5:1.

13. The humidity indicator array of claim 12, a copper(II) sulfate and sodium bromide react to produce the copper bromide.

14. The humidity indicator array of claim 12, wherein each area further includes cellulose fiber.

15. The humidity indicator array of claim 12, wherein the dye is an FD&C color.

16. The humidity indicator array of claim 12, wherein each of the plurality of areas of humidity indicators have increasing amounts of copper bromide and bromide salt and detect a humidity ranging from 10% to 50%.

17. The humidity indicator array of claim 16, wherein the plurality of areas of humidity indicators have a 10% separation from 10% to 50%.

18. The humidity indicator array of claim 13, wherein the weight ratio of sodium bromide to copper (II) sulfate pentahydrate ranges from about 3:1 to about 5:1.

19. The humidity indicator array of claim 13, wherein the weight ratio of sodium bromide to copper (II) sulfate pentahydrate is 3:1.

20. A method of humidity indication comprising:
providing a plurality of areas of humidity indication on an indicator card, wherein each area comprises copper bromide, dye, and bromide salt, wherein the mole ratio of bromide ions to copper ions in the area ranges from about 7.5:1 to 12.2:1;
bringing the indicator card into an environment containing humidity, whereby the areas of humidity indication below the humidity level change color;
reading a humidity as being at the highest color change area.

21. The method of claim 20, wherein the areas further include a cellulose fiber.

22. The method of claim 20, wherein the areas detect the humidity in 10% intervals in a range from 10%-50%.

23. The method of claim 20, wherein the dye is a FD&C yellow.

24. The method of claim 20, wherein the card is blotting paper.

25. A method of making a humidity indicator comprising:
providing a group of solutions of bromide salt, copper(II) sulfate, and dye; and
casting each solution onto a separate area of a substrate, wherein the solutions vary in amount of bromide and copper(II) sulfate and the dye amount Is substantially the same in each solution of the group.

26. The method of claim 25, wherein each area intended for sensing low humidity has a lower concentration of bromide salt and copper(II) sulfate.

27. The method of claim 25, wherein each area intended for sensing high humidity has a higher concentration of bromide salt and copper(II) sulfate.

28. The method of claim 25, wherein the substrate is blotting paper.

29. The method of claim 25, wherein the group of solutions includes cellulose fiber.

30. The method of claim 25, wherein the mole weight ratio of bromide to copper is about 7.5:1.

31. The method of claim 25, wherein the group of solutions detect a humidity ranging from 10% to 50%.

32. The method of claim 25, wherein the group of solutions detect a humidity in 10% intervals in a range of 10% to 50%.

33. A humidity indicator comprising a humidity indicator in a resin, the humidity indicator comprising copper bromide, bromide salt, and dye.

34. The humidity indicator of claim 33, further comprising a tablet, wherein the tablet comprises resin in powdered form pressed to form the tablet.

35. The humidity indicator of claim 33, wherein the resin is generally transparent.

36. The humidity indicator of claim 33, wherein the resin is a hot melt adhesive.

37. The humidity indicator of claim 33, wherein the humidity indicator is dispersed in the resin.

38. The humidity indicator of claim 33, wherein the humidity indicator is in at least one area of the resin.
